(19) United States
(12) Patent Application Publication
(10) Pub. No.: US 2019/0053759 A1
(43) Pub. Date: Feb. 21, 2019

(54) BIOSIGNAL SENSING PATCH AND BIOSIGNAL MONITORING DEVICE HAVING SAME

(71) Applicant: Samsung Electronics Co., Ltd., Suwon-si, Gyeonggi-do (KR)

(72) Inventors: Seong-je CHO, Suwon-si (KR); Seok-gin KANG, Suwon-si (KR); Jae-geol CHO, Yongin-si (KR); Sun-tae JUNG, Yongin-si (KR)

(21) Appl. No.: 15/770,597

(22) PCT Filed: Nov. 14, 2016
(86) PCT No.: PCT/KR2016/013058

§ 371 Date: Apr. 24, 2018

(30) Foreign Application Priority Data
Nov. 16, 2015 (KR) 10-2015-0160414

(51) Int. Cl. A61B 5/00; A61B 5/145

(52) U.S. Cl. CPC A61B 5/6833; A61B 5/14532; H04B 5/0031; A61B 5/0022; A61B 5/685

(57) ABSTRACT

The present invention relates to a biosignal monitoring device, comprising: a biosignal sensing patch which is attached to a skin and detects biosignals; and a monitoring patch which receives data sensed by the biosignal sensing patch and transmits the same to an external device, wherein the biosignal sensing patch comprises a sensor which is inserted into a skin and detects biosignals, a memory for storing data outputted from the sensor, and a short-range communication unit for receiving the data stored in the memory, and wherein the monitoring patch comprises an adjacent receiving unit for receiving data transmitted from the short-range communication unit of the biosignal sensing patch, and a transmission unit for transmitting the received data to an external device. In addition, the biosignal sensing patch transmits the data when the monitoring patch approaches or contacts the same, and the monitoring patch transmits, to an external device, the data received from the biosignal sensing patch.

### TECHNICAL FIELD

The present disclosure relates to a bio-signal sensing patch capable of sensing bio-signals by attaching to a skin of a human body, and more particularly, to a bio-signal sensing patch implemented so that that a patient can perform daily life without inconvenience in a state of being attached to the skin and a bio-signal monitoring device having the same.

### BACKGROUND ART

Various devices have been developed and widely used to continuously monitor bio-signals of patients according to the development of medical technology.

Particularly, diabetic patients need to constantly monitor and manage blood glucose levels while they are in their daily lives.

Conventionally, diabetic patients who personally monitor blood glucose levels have been used to periodically collect blood and determine blood glucose levels from the blood. However, this method is inconvenient because the patient must perform it by hand, the measured data are inconsistent, and there is a possibility that the patient will miss the measured data.

In order to solve this problem, a device provided with a sensor, which can measure blood glucose and be attached to a patient’ body, and capable of transmitting blood glucose data measured by the sensor to an external monitoring device has been developed and used.

In a device for monitoring blood glucose by using micro needles attached to the skin, a sensor including the micro needles and a transmitter for transmitting data measured by the sensor are integrally formed. At this time, the transmitter is generally heavier than the micro needles. Therefore, there is a problem that the micro needles may easily fall off from the skin even when a light impact is applied.

In addition, since the transmitter always operates to transmit the measured blood glucose data to the monitoring device in real time, the transmitter always consumes power. Therefore, there is a problem that it is necessary to use a battery having a large capacity for long-time use. When a battery having a small capacity is used to reduce the weight, there is a problem that the use time of such a device is reduced.

### DISCLOSURE OF INVENTION

The present disclosure has been developed in order to overcome the above drawbacks and other problems associated with the conventional arrangement. An aspect of the present disclosure relates to a bio-signal sensing patch that is attached to a skin, measures and stores bio-signals, and then transmits measured data only when necessary, in order to lighten a part always attached to the skin.

Another aspect of the present disclosure relates to a bio-signal monitoring device including a bio-signal sensing patch that is attached to a skin, measures and stores bio-signals and then transmits measured data only when necessary and a monitoring patch that receives data from the bio-signal sensing patch and transmits the data to an external device.

According to an aspect of the present disclosure, a bio-signal monitoring device may include a sensing patch configured to detect bio-signals and to store the detected bio-signals as data; a monitoring patch configured to receive the data when approaching the sensing patch; and an external device configured to receive the data received by the monitoring patch in real time.

The bio-signal sensing patch may include a sensor to be inserted into a skin and configured to detect the bio-signals; a memory configured to store the data output from the sensor; and a short range communicator configured to transmit the data stored in the memory.

The bio-signal sensing patch may include a sensor supporter supporting the sensor and to be in contact with the skin; a controller disposed on the sensor supporter and configured to control the memory to store the data output from the sensor; and a power supplier disposed on the sensor supporter and configured to supply power to the sensor, the memory, and the controller.

The sensor may include a micro needle array.

The short range communicator may include a near field communication (NFC) antenna.

The monitoring patch may include a short range communicator configured to receive the data transmitted from the bio-signal sensing patch and to transmit the received data to the external device.

The short range communicator may include a near field communication (NFC) antenna.

The short range communicator may include one of Bluetooth, wifi, and zigbee.

The monitoring patch may include a transmission memory configured to store the data transmitted from the bio-signal sensing patch; a transmission controller configured to control the short range communicator and the transmission memory to store and transmit the data; a power supplier configured to supply power to the transmission memory, the short range communicator, and the transmission controller; and a substrate on which the transmission memory, the short range communicator, the transmission controller, and the power supplier are disposed.

The monitoring patch may include a display configured to display the data.

In accordance with another aspect, a bio-signal sensing patch may include a sensor to be inserted into a skin and configured to detect bio-signals; a sensor supporter supporting the sensor; a memory disposed on the sensor supporter and configured to store data output from the sensor; a controller disposed on the sensor supporter and configured to control the memory to store the data output from the sensor; a short range communicator disposed on the sensor supporter and configured to transmit the data stored in the memory to a reader; and a power supplier disposed on the sensor supporter and configured to supply power to the sensor, the memory, and the controller, wherein the short range communicator may transmit the data when the reader approaches the short range communicator.

The short range communicator may include a near field communication (NFC) antenna.

The power supplier may include a film type battery.

The sensor may include a micro needle array.

The micro needle array may include comprises a plurality of micro needles, the plurality of micro needles may be formed of a shape memory alloy, and when the micro needles are inserted into the skin, distal end portions of the micro needles may be bent obliquely with respect to an inserting direction of the micro needles and prevent the micro needles from falling off from the skin.

The micro needle array may include a plurality of micro needles, the plurality of micro needles may be formed of a bimetal, and when the micro needles are inserted into the skin, the micro needles may be bent obliquely with respect to an inserting direction of the micro needles and prevent the micro needles from falling off from the skin.

The sensor supporter may include at least one elastic bend portion provided on both sides of the micro needle array and defining a protrusion height of the micro needle array.

The at least one elastic bend portion may be formed of a plate spring.

The bio-signal sensing patch may include a needle protection cover provided below the micro needle array.

The micro needle array may be configured to adjust interval between the plurality of micro needles.

The reader may include a monitoring patch. The monitoring patch may include a transmission memory configured to store the data transmitted from the bio-signal sensing patch; a short range receiver configured to receive the data from the short range communicator of the bio-signal sensing patch; a transmitter configured to transmit the data stored in the memory; a transmission controller configured to control the short range receiver, the memory, and the transmitter to store and transmit the data; a power supplier configured to supply power to the transmission memory, the short range receiver, the transmitter, and the transmission controller; and a substrate on which the transmission memory, the short range receiver, the transmitter, the transmission controller, and the power supplier are disposed.

The reader may include a smartphone.

### BRIEF DESCRIPTION OF DRAWINGS

FIG. 1 is a view schematically illustrating a bio-signal monitoring device according to an embodiment of the present disclosure disposed in a patient's arm;

FIG. 2 is a view conceptually illustrating a bio-signal monitoring device according to an embodiment of the present disclosure;

FIG. 3 is a functional block diagram of a bio-signal monitoring device according to an embodiment of the present disclosure;

FIG. 4A is a view illustrating an example of using a bio-signal monitoring device according to an embodiment of the present disclosure during daily living;

FIG. 4B is a view illustrating an example of using a bio-signal monitoring device according to an embodiment of the present disclosure when sleeping;

FIG. 5A is a partial view illustrating an example of micro needles of a bio-signal sensing patch an according to an embodiment of the present disclosure;

FIG. 5B is a partial view illustrating a case where the micro needles of FIG. 5A are inserted into a skin;

FIG. 6A is a partial view illustrating another example of micro needles of a bio-signal sensing patch an according to an embodiment of the present disclosure;

FIG. 6B is a partial view illustrating a case where the micro needles of FIG. 6A are inserted into a skin;

FIG. 7 is a perspective view illustrating an example of a micro needle array used in a bio-signal sensing patch according to an embodiment of the present disclosure;

FIG. 8 is a view illustrating transformation steps of the micro needle array of FIG. 7;

FIG. 9A is a view illustrating a state before the micro needle array of FIG. 7 is inserted into a skin;

FIG. 9B is a view illustrating a state where the micro needle array of FIG. 7 is inserted into a skin;

FIG. 10 is a perspective view illustrating an example of a micro needle array used in a bio-signal sensing patch according to an embodiment of the present disclosure;

FIG. 11A is a perspective view illustrating a bio-signal sensing patch according to an embodiment of the present disclosure having a protection cover;

FIG. 11B is a view illustrating a case where the protection cover of the bio-signal sensing patch of FIG. 11A is opened; and

FIG. 12 is a perspective view illustrating an example of a micro needle array used in a bio-signal sensing patch according to an embodiment of the present disclosure.

### BEST MODE FOR CARRYING OUT THE INVENTION

Hereinafter, embodiments of a bio-signal sensing patch according to the present disclosure and a bio-signal monitoring device having the same will be described in detail with reference to the accompanying drawings.

It is to be understood that the embodiments described below are provided for illustrative purpose only, and that the present disclosure may be embodied with various modifications different form exemplary embodiments described herein. However, in the following description below, detailed description of well-known functions or components will be omitted when it may be unnecessary to obscure the subject matter of the present disclosure. Further, the accompanying drawings may be not drawn to scale in order to facilitate understanding of the invention, but the dimensions of some of the components may be exaggerated.

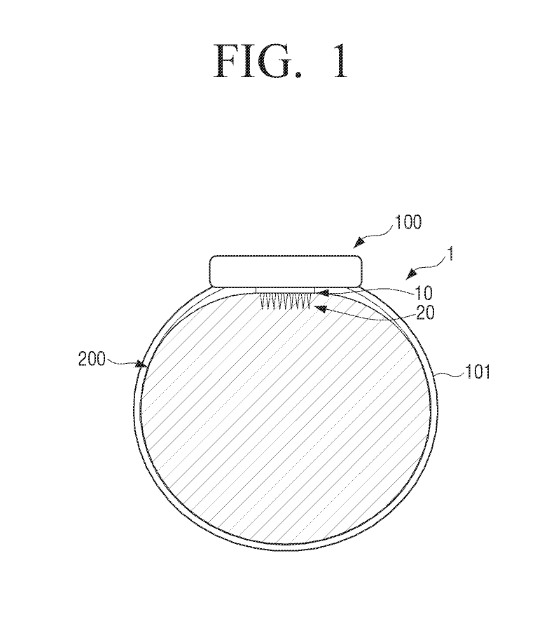

Hereinafter, a bio-signal monitoring device according to an embodiment of the present disclosure will be described with reference to FIGS. 1 to 3.

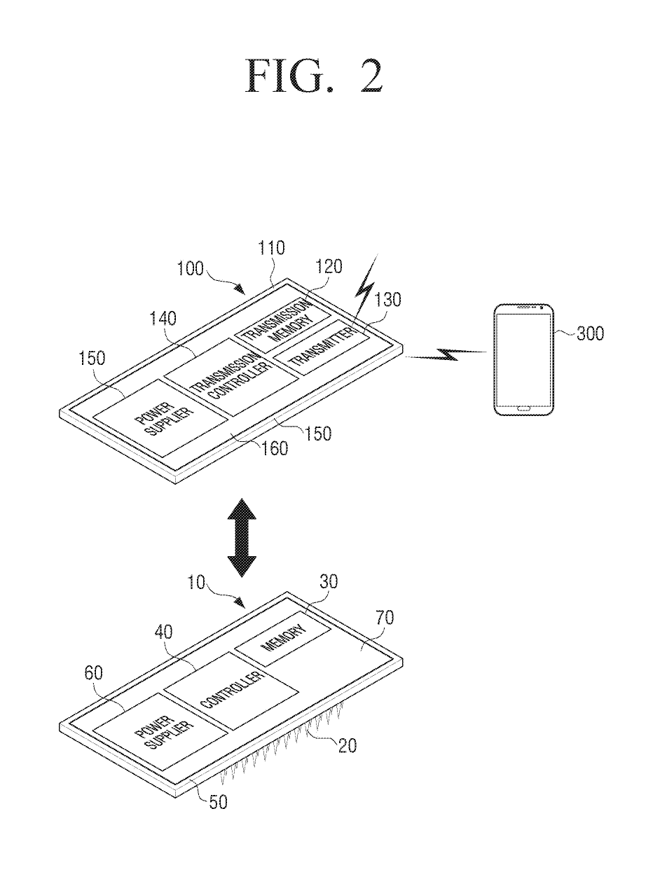

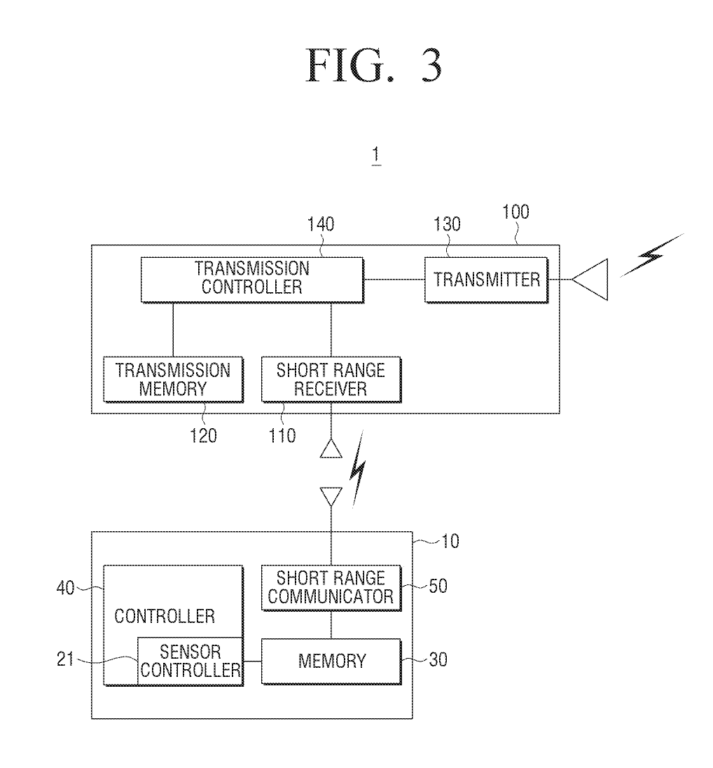

FIG. 1 is a view schematically illustrating a bio-signal monitoring device according to an embodiment of the present disclosure disposed in a patient's arm. FIG. 2 is a view conceptually illustrating a bio-signal monitoring device according to an embodiment of the present disclosure, and FIG. 3 is a functional block diagram of a bio-signal monitoring device according to an embodiment of the present disclosure.

Referring to FIGS. 1 to 3, a bio-signal monitoring device 1 according to an embodiment of the present disclosure may include a bio-signal sensing patch 10 and a monitoring patch 20.

The bio-signal sensing patch 10 may include a sensor 20, a memory 30, a controller 40, a short range communicator 50, a power supplier 60, and a sensor supporter 70.

The sensor 20 is inserted into a skin of a patient to detect bio-signals and includes a micro needle array 22 and a sensor controller 21. In the case of the present embodiment, the sensor 20 is formed to detect the glucose concentration of the patient.

The micro needle array 22 may include a plurality of micro needles 23 arranged in a predetermined pattern. In a state where the micro needles 23 are inserted into the vicinity of the upper portion of the dermal layer of the patient’ skin (at a depth of about 0.5 to 0.9 mm from the skin surface), the sensor controller 21 measures the concentration of glucose by applying minute electricity to the micro needle array 22 and detecting an amount of electricity distributed around the micro needle array 22. The micro needle array 22 according to an embodiment of the present disclosure is configured not to be separated from the skin. A specific structure for preventing the micro needle array 22 from being separated from the skin will be described in detail below.

A memory 30 stores data related to the bio-signals measured by the sensor 20, in this embodiment, the glucose concentration.

The short range communicator 50 transmits data stored in the memory 30 when the monitoring patch 100 approaches the bio-signal sensing patch 10, for example, when the monitoring patch 100 comes adjacent to or into contact with the sensing patch. The short range communicator 50 may be formed to transmit data stored in the memory 30 only when the monitoring patch 100 approaches within about 10 cm of or comes into contact with the bio-signal sensing patch 10. Accordingly, the short range communicator 50 may be implemented as a short range transmitter having only a transmission function at a near distance.

As an example, the memory 30 and the short range communicator 50 may be implemented by a near field communication (NFC) method. In other words, the memory 30 may be included in an NFC chip, and the short range communicator 50 may be implemented as an NFC antenna. Therefore, the glucose concentration data measured by the sensor 20 is stored in the NFC chip 30. When the monitoring patch 100 approaches the bio-signal sensing patch 10 within 10 cm, the data stored in the NFC chip 30 is transmitted. In other words, the memory 30 and the short range communicator 50 may be formed to function as an NFC tag.

The controller 40 is configured to store the data measured by the sensor 20 in the memory 30. For example, the controller 40 controls the sensor 20 to measure the glucose concentration at predetermined time intervals, and stores the data on the glucose concentration measured by the sensor 20 in the memory 30. The controller 40 may be formed integrally with the sensor controller 21 of the sensor 20. For example, the sensor controller 21 may be configured as a part of the controller 40.

The power supplier 60 is configured to supply power to the controller 40, the sensor 20, and the memory 30. A battery may be used as the power supplier 60. In the case of the present embodiment, a film-type battery is used. In the case of the present disclosure, the electricity consumption of the power supplier 60 may be reduced because no separate electricity supply is required when the data stored in the memory 30 is transmitted to the outside. Therefore, the power supplier 60 may be used for a longer period of time than the conventional technique in which the sensor 20 continuously transmits measured data.

The sensor supporter 70 may be formed to fix and support the sensor 20, the memory 30, the controller 40, the power supplier 60, and the short range communicator 50. For example, the sensor supporter 70 may be formed of a flexible printed circuit board. At this time, the micro needle array 22 of the sensor 20 is provided on the bottom surface of the flexible printed circuit board 70, and the sensor controller 21, the memory 30, the controller 40, the power supplier 60, and the short range communicator 50 may be provided on the top surface of the flexible printed circuit board 70.

As another example, the micro needle array 22 of the sensor 20, the sensor controller 21, the memory 30, the controller 40, the power supplier 60, and the short range communicator 50 may be provided on the same side of the flexible printed circuit board 70. In this case, the sensor controller 21, the memory 30, the controller 40, the power supplier 60, and the short range communicator 50 may be disposed around the micro needle array 22.

As another example, some of the sensor controller 21, the memory 30, the controller 40, the power supplier 60, and the short range communicator 50 may be provided on the top surface of the flexible printed circuit board 70, and the remaining parts may be provided on the bottom surface of the flexible printed circuit board 70 in the same manner as the micro needle array 22.

The monitoring patch 100 may be configured to receive data transmitted from the bio-signal sensing patch 10 and transmit the received data to an external device 300 located at a relatively long distance. For example, the monitoring patch 100 may be formed to receive data transmitted from the bio-signal sensing patch 10 when the monitoring patch 100 approaches the bio-signal sensing patch 10 within 10 cm and to transmit the received data to the external device 300 that is disposed within about 10 m and spaced apart about 10 cm or more from the bio-signal sensing patch 10. In addition, the monitoring patch 100 may be formed to be optionally disposed adjacent to or separated from the bio-signal sensing patch 10.

The monitoring patch 100 may include a short range communicator, a transmission memory 120, a transmission controller 140, and a power supplier 150.

The short range communicator may include a short range receiver 110 and a transmitter 130. The short range receiver 110 is configured to receive data from the short range communicator 50 of the bio-signal sensing patch 10. The short range receiver 110 receives data stored in the memory 30 of the bio-signal sensing patch 10 from the short range communicator 50 of the bio-signal sensing patch 10 when the monitoring patch 100 approaches or contacts the bio-signal sensing patch 10.

The transmission memory 120 is formed to store data transmitted from the bio-signal sensing patch 10.

As an example, the short range receiver 110 and the transmission memory 120 may be implemented as a near field communication (NFC) reader. In detail, the short range receiver 110 may be formed as an NFC antenna, and the transmission memory 120 may be formed as an NFC chip. Therefore, when the monitoring patch 100 is in contact with the bio-signal sensing patch 10 or is adjacent to the bio-signal sensing patch 10 within 10 cm, the NFC chip 120 may receive and store the data stored in the memory 30 of the bio-signal sensing patch 10 through the NFC antenna 110.

The transmitter 130 is configured to transmit data stored in the transmission memory 120 to the outside. In detail, the transmitter 130 may be configured to transmit the data to the external device 300 that is located at a distance greater than the distance that the short range receiver 110 of the monitoring patch 100 and the short range communicator 50 of the bio-signal sensing patch 10 can communicate with each other, for example, an analyzer or a smartphone that is located 10 cm or more and 10 meters or less from where the bio-signal sensing patch 10 is located. The transmitter 130 may be a Bluetooth, a wifi, a zigbee, or the like.

The transmitter 130 may transmit the data received from the bio-signal sensing patch 10 to the smartphone 300. In this case, the smartphone 300 needs to have a Bluetooth, a wifi, a zigbee, or the like capable of bidirectional communication with the transmitter 130. Also, the smartphone 300 may be provided with an analysis program for analyzing the received glucose concentration data and displaying the analysis result.

The transmission controller 140 may control the transmission memory 120 and the transmitter 130 to transmit the data stored in the transmission memory 120 to the external analysis device in real time.

In addition, when the monitoring patch 100 is configured to receive the data from the bio-signal sensing patch 10 through the NFC method, the transmission controller 140 may turn on/off the power of the monitoring patch 100 at predetermined time intervals. In the NFC communication, data are transmitted and received only when a part for transmitting data (NFC tag) and a part for receiving the data (NFC reader) are adjacent to or in contact with each other. Therefore, when the monitoring patch 100 is brought adjacent to or into contact with the bio-signal sensing patch 10, the data transmission/reception is performed only at first approach and thereafter the data transmission/reception does not occur so that the monitoring patch 100 cannot continuously receive the data. Accordingly, it is necessary to turn on/off the power of the monitoring patch 100 in order to achieve the same effect as repeatedly performing the operation of attaching and separating the monitoring patch 100 to and from the bio-signal sensing patch 10. Therefore, when the power of the monitoring patch 100 is turned on/off at predetermined time intervals, the monitoring patch 100 may receive data from the bio-signal sensing patch 10 at the predetermined time intervals and continuously transmit the data to the external device 300.

The power supplier 150 is configured to supply power to the transmission memory 120, the transmitter 130, and the transmission controller 140. In the present disclosure, since the power supplier 150 of the monitoring patch 100 is formed separately from the bio-signal sensing patch 10 having the micro needle array 22, a battery having a large capacity may be used as the power supplier 150 so that the monitoring patch 100 may be used for a long time.

The transmission memory 120, the short range receiver 110, the transmitter 130, the transmission controller 140, and the power supplier 150 may be disposed on a substrate 160. The substrate 160 may be formed of a flexible printed circuit board. The substrate 160 on which the transmission memory 120, the short range receiver 110, the transmitter 130, the transmission controller 140, and the power supplier 150 are disposed may be housed in a housing 170 so that these components are not exposed to the outside.

The housing 170 may be fixed to the skin 200 of a patient by a fixing member 101. As the fixing member 101, a band, an adhesive tape, or the like may be used. In addition, various methods may be used as the fixing member 101 as long as they can fix the housing 170 to the skin 200.

The monitoring patch 100 may be fixed to the skin 200 of the patient so as to be positioned just above the bio-signal sensing patch 10 as illustrated in FIG. 1. However, this is only an embodiment, and the monitoring patch 100 may be disposed on the skin 200 of the patient at a certain distance from the bio-signal sensing patch 10. The monitoring patch 100 may be disposed within a distance where the short range receiver 110 can communicate with the short range communicator 50 of the bio-signal sensing patch 10.

As another embodiment, the monitoring patch 100 may be configured to include a display part (not illustrated) capable of displaying received data. In addition, the monitoring patch 100 may further include an alarm part (not illustrated) that can inform the patient when hypoglycemia occurs.

In FIGS. 1 to 3, the glucose concentration data measured by the sensor 20 of the bio-signal sensing patch 10 are transmitted to the external analysis device such as the smartphone 300 through the monitoring patch 100. However, the present disclosure is not limited thereto.

The present disclosure may use the smartphone 300 directly as a reader instead of the monitoring patch 100 functioning as a reader for reading data of the bio-signal sensing patch 10.

At this time, the smartphone 300 may include a short range receiver capable of communicating with the bio-signal sensing patch 10 and a memory capable of storing the received data. In detail, the smartphone 300 includes an NFC chip and an NFC antenna capable of performing NFC communication. The smartphone 300 is also provided with an analysis program or application for analyzing and displaying the glucose concentration data received from the bio-signal sensing patch 10.

In the case where the bio-signal sensing patch 10 provided with the sensor 20 and the monitoring patch 100 configured to receive and transmit the glucose concentration data are formed separately as in the present disclosure, the present disclosure may be used variously as follows.

When the patient can feel his/her blood glucose status as in the daytime, only the bio-signal sensing patch 10 is attached to the skin 200, and if necessary, the glucose concentration may be confirmed by bringing the monitoring patch 100 or the smartphone 300 close to the bio-signal sensing patch 10.

On the other hand, since the hypoglycemia alarm function is required at night when the patient takes sleep, the monitoring patch 100 is fixed to the skin 200 of the patient in the vicinity of the bio-signal sensing patch 10 as illustrated in FIG. 1. Thus, the monitoring patch 100 receives data from the bio-signal sensing patch 10 and transmits the data to the smartphone 300 or the external analysis device, so that when hypoglycemia occurs, the smartphone 300 or the external analysis device generates an alarm to inform the patient.

Further, in the case where only the sensor 20, the memory 30, and the short range communicator 50 are disposed in the bio-signal sensing patch 10 attached to the patient's body as in the present disclosure, a battery having a small capacity may be used as the power supplier 60 because the required amount of electricity is small. Therefore, according to the present disclosure, cost of the bio-signal sensing patch 10 to be discarded after a predetermined period of use may be reduced.

In addition, in the case where only the sensor 20, the memory 30, and the short range communicator 50 are provided in the bio-signal sensing patch 10 attached to the patient's body as in the present disclosure, the weight of the bio-signal sensing patch 10 may be minimized. Therefore, when the bio-signal sensing patch 10 is attached to the patient’ skin 200, the separation of the micro needle array 22 from the skin may be suppressed as much as possible.

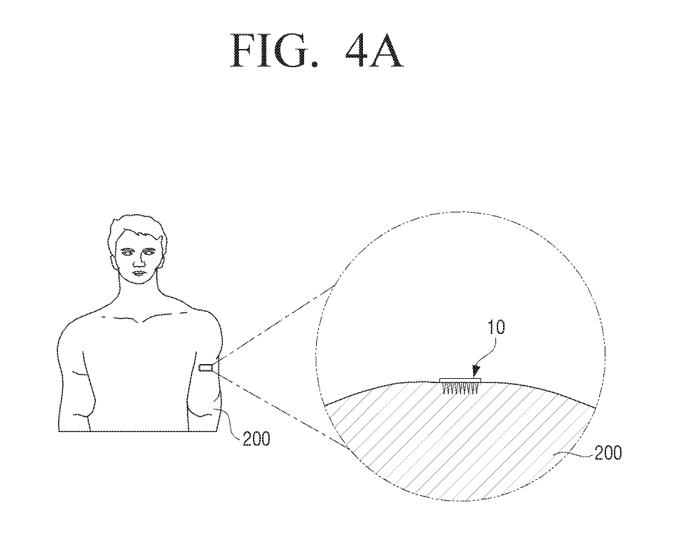

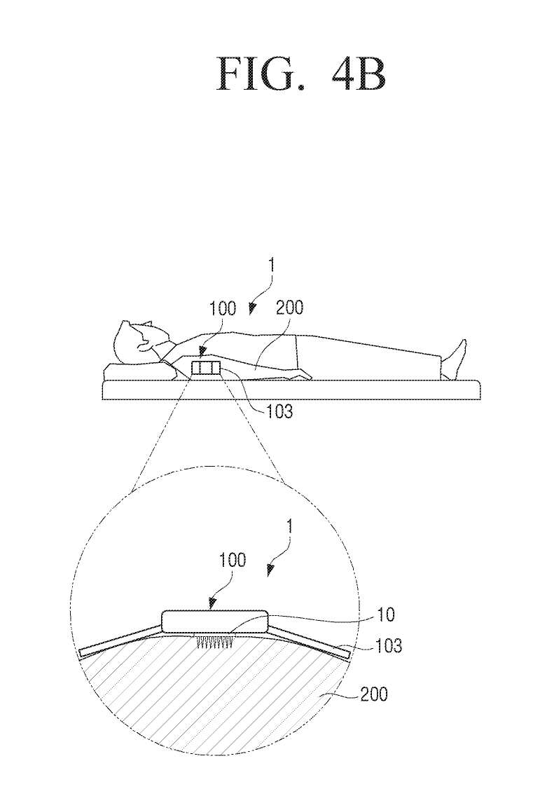

Hereinafter, a use of the bio-signal monitoring device 1 according to an embodiment of the present disclosure will be described in detail with reference to FIGS. 4A and 4B.

FIG. 4A is a view illustrating an example of using a bio-signal monitoring device according to an embodiment of the present disclosure during daily living, and FIG. 4B is a view illustrating an example of using a bio-signal monitoring device according to an embodiment of the present disclosure when sleeping. For reference, in FIGS. 4A and 4B, a partial cross-sectional view schematically illustrating a state where a bio-signal sensing patch and a bio-signal monitoring device are disposed is shown in a circle.

During daily living, as illustrated in FIG. 4A, the user wears or attaches only the bio-signal sensing patch 10 to be in contact with the skin 200. When necessary, the user brings the smartphone close to the bio-signal sensing patch 10 or contacts the smartphone with the bio-signal sensing patch 10. Then, the smartphone receives the bio-signal data from the bio-signal sensing patch 10 using the NFC communication function and stores the data in the memory of the smartphone. Thereafter, the user may analyze the received bio-signal data using the data analysis program or application installed in the smartphone to grasp the current state of the user.

When sleeping, the user cannot feel his/her body condition. Accordingly, as illustrated in FIG. 4B, the user combines the monitoring patch 100 with the bio-signal sensing patch 10 disposed on the skin 200 and takes a sleep while wearing the monitoring patch 100 on his/her body. At this time, the monitoring patch 100 may be coupled to the bio-signal sensing patch 10 by various methods. FIG. 4 illustrates a case where the monitoring patch 100 is attached to the user's skin 200 using an adhesive tape 103. Alternatively, the monitoring patch 100 may be configured in the form of a band 101 to wrap the bio-signal sensing patch 10 as illustrated in FIG. 1.

Then, the monitoring patch 100 receives bio-signal data from the bio-signal sensing patch 10 at predetermined time intervals and transmits the bio-signal data to the external device such as a smartphone in real time. Then, the external device analyzes the received bio-signal data in real time. When a health problem such as hypoglycemia occurs, the external device may generate an alarm, thereby warning the patient or the person around him or her.

Hereinafter, the structure of the micro needle array 22 constituting the bio-signal sensing patch 10 to prevent the micro needle array 22 from falling off from the skin 200 will be described in detail with reference to the accompanying drawings.

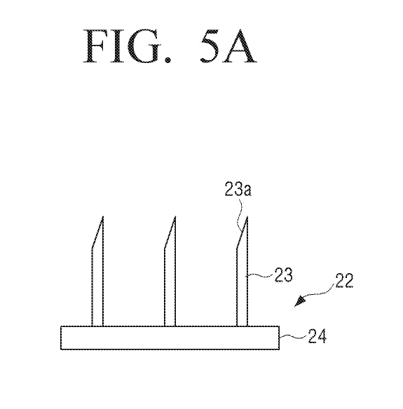

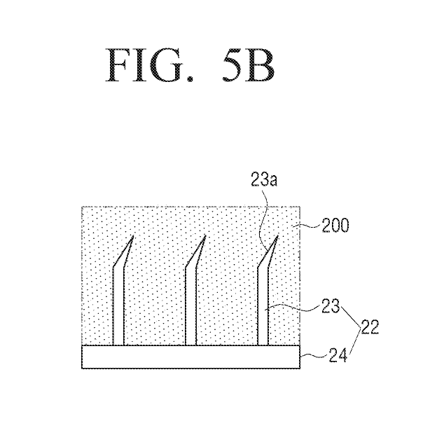

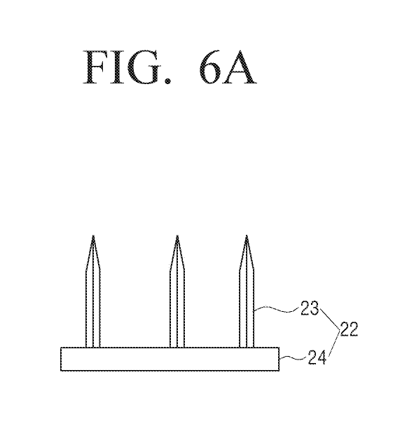

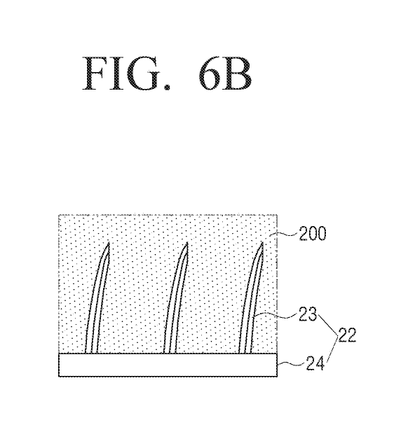

FIGS. 5A to 6B illustrates a case where the micro needles themselves are configured not to fall off from the skin.

FIG. 5A is a partial view illustrating an example of micro needles of a bio-signal sensing patch an according to an embodiment of the present disclosure, and FIG. 5B is a partial view illustrating a case where the micro needles of FIG. 5A are inserted into a skin. FIG. 6A is a partial view illustrating another example of micro needles of a bio-signal sensing patch an according to an embodiment of the present disclosure, and FIG. 6B is a partial view illustrating a case where the micro needles of FIG. 6A are inserted into a skin.

Distal end portions of the plurality of micro needles 23 constituting the micro needle array 22 may be formed of a shape memory alloy. At this time, the distal end portions 23a of the micro needles 23 formed of the shape memory alloy may be formed so that the distal end portion 23a of the micro needle 23 is inclined at a predetermined angle with respect to the longitudinal direction of the micro needle 23 as illustrated in FIG. 5B at a temperature similar to a human body temperature, for example, a temperature range of 35° C. to 38° C. and is restored to an original state in which the distal end portion 23a of the micro needle 23 is perpendicular to a base 24 as illustrated in FIG. 5A when the temperature becomes lower than the human body temperature.

Therefore, before the micro needle array 22 is inserted into the skin 200, the distal end portions 23a of the micro needles 23 remain vertical as illustrated in FIG. 5A. When the micro needle array 22 is inserted into the skin 200, the distal end portions 23a of the micro needles 23 made of the shape memory alloy are bent obliquely with respect to the longitudinal direction of the micro needles 23, that is, the inserting direction of the micro needles 23 as illustrated in FIG. 5B. When the distal end portions 23a of the micro needles 23 are bent in an inclined manner, the micro needles 23 do not fall off the skin 200 easily. Therefore, the bio-signal sensing patch 10 provided with the micro needle array 22 does not fall off the skin 200 easily.

In the case where the bio-signal sensing patch 10 is to be removed from the skin 200, when the temperature of the micro needle array 22 is lowered, the distal end portions 23a of the micro needles 23 are straightened, so that the micro needle array 22 may be easily removed from the skin 200.

As another embodiment, the micro needles 23 may be formed of a bimetal. At this time, the micro needles 23 formed of the bimetal may be formed so that the micro needle 23 is inclined at a predetermined angle with respect to a base 24, that is, inclined at the predetermined angle with respect to the inserting direction of the micro needles 23 as illustrated in FIG. 6B at a temperature similar to the human body temperature, for example, a temperature range of 35° C. to 38° C. and is restored to an original state in which the micro needle 23 are perpendicular to the base 24 as illustrated in FIG. 6A when the temperature becomes lower than the human body temperature.

Therefore, before the micro needle array 22 is inserted into the skin 200, the micro needles 23 remain perpendicular to the base 24 as illustrated in FIG. 6A. When the micro needle array 22 is inserted into the skin 200, the micro needles 23 made of the bimetal are bent obliquely with respect to the longitudinal direction of the micro needles 23, that is, the inserting direction of the micro needles 23 as illustrated in FIG. 6B. When the micro needles 23 are bent in an inclined manner, the micro needles 23 do not fall off the skin 200 easily. Therefore, the bio-signal sensing patch 10 provided with the micro needle array 22 does not fall off the skin 200 easily.

In the case where the bio-signal sensing patch 10 is to be removed from the skin 200, when the temperature of the micro needle array 22 is lowered, the micro needles 23 are straightened to be perpendicular to the base 24, so that the micro needle array 22 may be easily removed from the skin 200.

Hereinafter, a case where an elastic bend portion is formed on the base of the micro needle array so that the micro needles are not easily removed from the skin will be described.

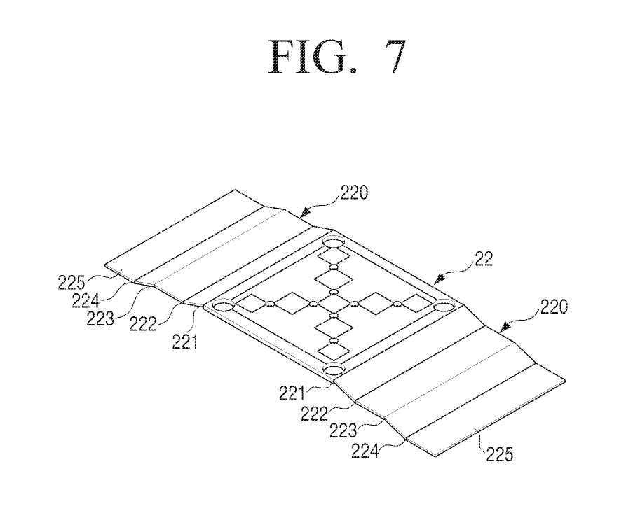

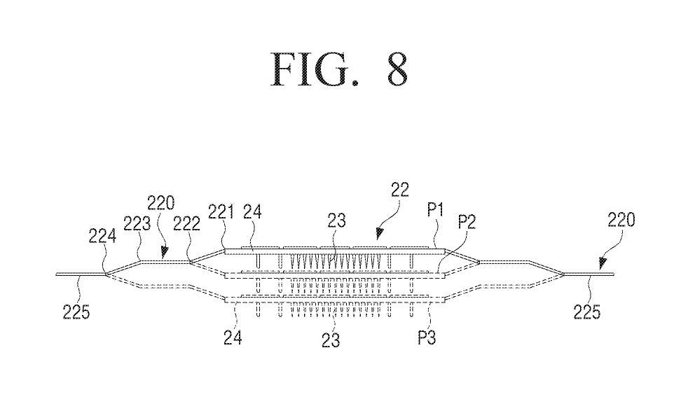

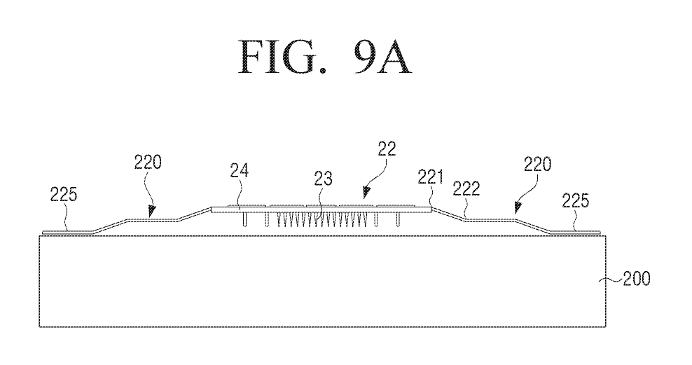

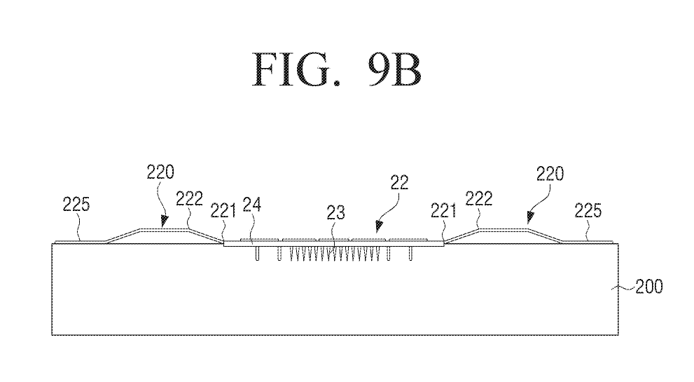

FIG. 7 is a perspective view illustrating an example of a micro needle array used in a bio-signal sensing patch according to an embodiment of the present disclosure. FIG. 8 is a view illustrating transformation steps of the micro needle array of FIG. 7. FIG. 9A is a view illustrating a state before the micro needle array of FIG. 7 is inserted into a skin, and FIG. 9B is a view illustrating a state where the micro needle array of FIG. 7 is inserted into the skin. For reference, in FIGS. 7 to 9B, the sensor supporter provided on the top surface of the micro needle array is not illustrated in order to clearly show an elastic supporter of the micro needle array.

Referring to FIG. 7, the micro needle array 22 includes elastic supporters 220 formed on both sides thereof. The elastic supporters 220 are formed to apply a predetermined force to the micro needle array 22 to prevent the micro needles 23 from falling off from the skin due to the elastic force of the skin. The elastic supporters 220 may be formed to act like a plate spring. For example, the elastic supporters 220 may be formed to have at least one elastic bend portion 221, 222, 223, and 224. The elastic bend portions 221, 222, 223, and 224 may be bent so as to bend at a predetermined angle. Therefore, the micro needle array 22 may be stably positioned at one of two stable positions when a predetermined force is applied to the micro needle array 22. When the micro needle array 22 in the stable position is applied with a force smaller than the force which can escape the micro needle array 22 from the stable position of the elastic bend portions 221, 222, 223, and 224, a repulsive force preventing the micro needle array 22 from escaping from the stable position is generated by the elastic supporters 220. Therefore, the micro needle array 22 may stably maintain a state inserted into the skin 200 even when a force is applied by the elastic force of the skin. For this purpose, the elastic supporters 220 may be formed by bending a metal plate having elasticity.

In the embodiment illustrated in FIG. 7, the elastic supporter 220 includes four elastic bend portions 221, 222, 223, and 224 formed to have step differences. When the elastic supporter 220 is formed to have three steps and four elastic bend portions 221, 222, 223, and 224 as described above, the micro needle array 22 supported by the elastic supporters 220 has three stable positions. In other words, the height at which the micro needle array 22 protrudes from the fixed ends 225 of the elastic supporters 220 is determined by the elastic supporters 220.

The three stable positions P1, P2, and P3 where the micro needle array 22 may be positioned by the elastic supporters 220 as illustrated in FIG. 7 are illustrated in FIG. 8.

In the first stable position P1, the leading end of the micro needle array 22 is positioned at a position higher than the fixed ends 225 of the elastic supporters 220. In this case, since the micro needle array 22 does not protrude from the elastic supporters 220, the leading end of the micro needle array 22 may be prevented from being damaged. When the micro needle array 22 is on the same plane as the fixed ends 225, the leading end of the micro needle array 22 protrudes so that the micro needles 23 may be damaged and the user may be hurt by the micro needles 23. Therefore, when the micro needle array 22 is handled in a state where the micro needle array 22 is in the first stable position P1, the above-described danger may be avoided.

A second stable position P2 is a case where the fixed ends 225 of the elastic supporters 220 and the base 24 of the micro needle array 22 are located on the same plane and the plurality of micro needles 23 protrude from the fixed ends 225. In this case, the micro needle array 22 is inserted into the skin. In this state, the micro needle array 22 is positioned in the stable position by the elastic bend portions 221, 222, 223, and 224 of the elastic supporters 220, so that when a force for separating the micro needle array 22 from the skin is applied to the micro needle array 22 by the elastic force of the skin, the micro needle array 22 may maintain a state in which the micro needle array 22 is stably attached to the skin due to the restoring force applied to the micro needle array 22 by the elastic supporters 220.

In a third stable position P3, the base 24 of the micro needle array 22 is positioned below the fixed ends 225 of the elastic supporters 220. The third stable position P3 may be used when the position into which the micro needle array 22 is inserted is deeper than the fixed ends 225 of the elastic supporters 220.

The case where the micro needle array 22 as illustrated in FIG. 7 is inserted into the skin will be described with reference to FIGS. 9A and 9B.

Before the micro needle array 22 is inserted into the skin 200, the micro needle array 22 is in a state as illustrated in FIG. 9A. In other words, the fixed ends 225 of the elastic supporters 220 are in contact with the skin 200 and the micro needle array 22 is spaced apart from the skin 200 so that the distal ends of the micro needles 23 are not in contact with the skin 200.

At this time, when the top surface of the micro needle array 22 is applied with a predetermined force, that is, a force that can overcome the restoring force of the elastic supporters 220, as illustrated in FIG. 9B, the first and second bend portions 221 and 222 of the elastic supporters 220 are bent so that the base 24 of the micro needle array 22 is brought into contact with the skin 200 and the plurality of micro needles 23 are inserted into the skin 200. At this time, since the micro needle array 22 is located at the second stable position P2, even when a force in the opposite direction is applied to the micro needle array 22 by the elastic force of the skin 200, the micro needle array 22 may stably maintain a state in which the micro needle array 22 is inserted into the skin due to the restoring force of the elastic supporters 220.

As described above, when the elastic supporters 220 are formed to have the plurality of stable positions P1, P2, and P3, the insertion depth of the micro needles 23 may be adjusted by the stable positions and the insertion of the micro needles 23 may be stably maintained at each insertion depth.

In the above description, the elastic supporters 220 are formed so that the micro needle array 22 has three stable positions P1, P2, and P3. However, the elastic supporters 220 may be formed so that the micro needle array 22 has two stable positions or four or more stable positions.

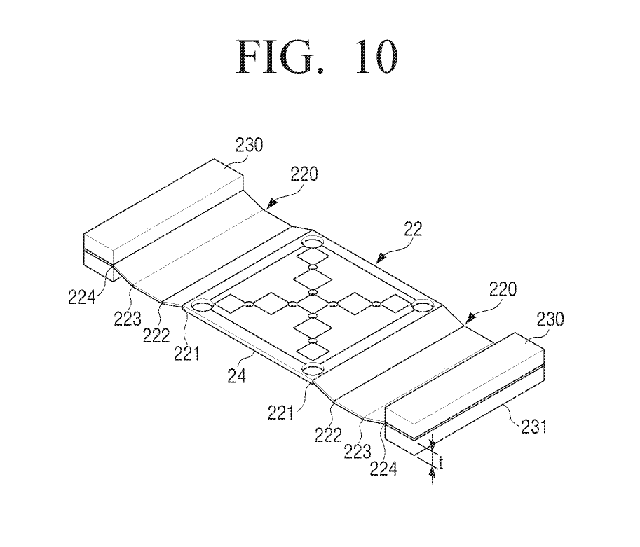

Hereinafter, a case in which a micro needle array according to an embodiment of the present disclosure is mounted on housings will be described with reference to FIG. 10.

FIG. 10 is a perspective view illustrating an example of a micro needle array used in a bio-signal sensing patch according to an embodiment of the present disclosure.

Referring to FIG. 10, the micro needle array 22 may include elastic supporters 220 provided on both sides thereof. The elastic supporters 220 include a plurality of elastic bend portions 221, 222, 223, and 224 to adjust the height of the micro needle array 22.

When the elastic supporters 220 are formed to have the plurality of elastic bend portions 221, 222, 223, and 224, it may be prevented that the micro needles 23 do not or partially protrude out of the housing 230 due to the thickness of the housing 230 when the flat micro needle array 22 is mounted on the housings 230.

In other words, when the elastic supporters 220 are bent to fit the thickness t of the housing 230, the base 24 of the micro needle array 22 may be aligned with the outer surface 231 of the housing 230.

Alternatively, when the elastic bend portions 221, 222, 223, and 224 capable of adjusting the height of the micro needle array 22 are additionally formed as illustrated in FIG. 10, the micro needle array 22 may be provided with two or more stable positions as described above. The micro needle array 22 illustrated in FIG. 10 is formed to have two stable positions.

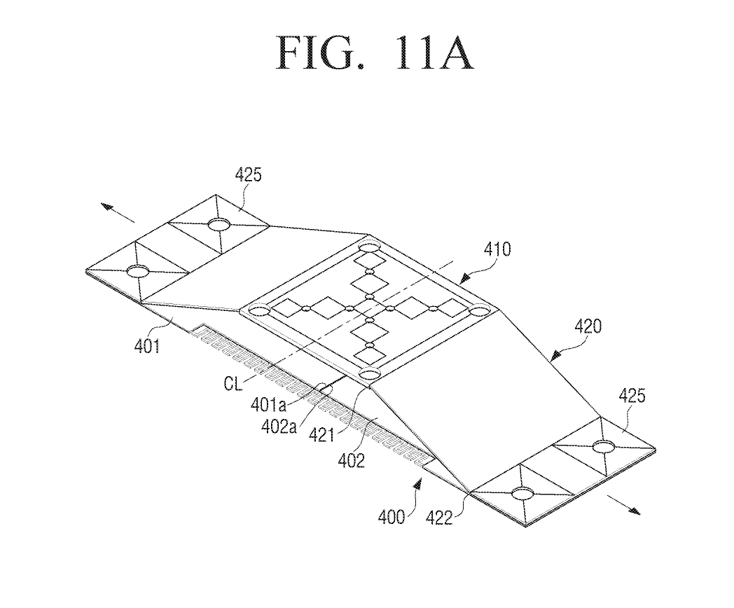

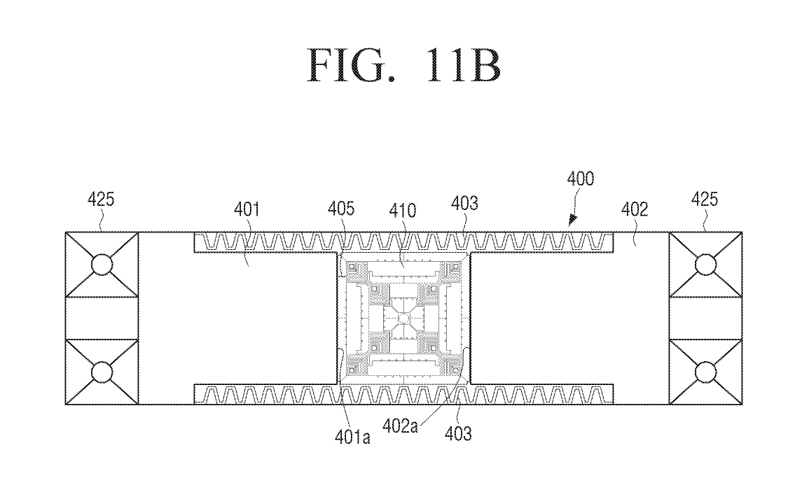

Hereinafter, a bio-signal sensing patch having a needle protection cover that can prevent micro needles of a micro needle array from being exposed will be described with reference to FIGS. 11A and 11B.

FIG. 11A is a perspective view illustrating a bio-signal sensing patch according to an embodiment of the present disclosure having a protection cover, and FIG. 11B is a view illustrating a case where the protection cover of the bio-signal sensing patch of FIG. 11A is opened. For reference, for the sake of convenience of illustration and explanation, the sensor supporter provided on the top surface of the micro needle array is omitted in FIG. 11A.

Referring to FIG. 11A, a needle protection cover 400 is provided below a micro needle array 410 of a bio-signal sensing patch according to an embodiment of the present disclosure.

The micro needle array 410 is provided with elastic supporters 420 having two bend portions 421 and 422 on both sides of the micro needle array 410. The micro needle array 410 is spaced upward from fixed ends 425 of the elastic supporters 420.

The needle protection cover 400 includes two cover members 401 and 402 formed in a planar shape. The two cover members 401 and 402 are formed symmetrically with respect to the center line CL of the micro needle array 410. When a force is applied to the micro needle array 410, the two cover members 401 and 402 are moved away from the center line CL of the micro needle array 410 to form an opening 405 through which the micro needle array 410 is exposed.

For example, a first cover member 401 is fixed to the left fixed end 425 of the elastic supporters 420 and a second cover member 402 is fixed to the right fixed end 425 of the elastic supporters 420 as illustrated in FIG. 11A. One end of the first cover member 401 and one end of the second cover member 402 are disposed to be in contact with each other at the center of the micro needle array 410. Further, the first and second cover members 401 and 402 are elastically supported by a pair of springs 403 provided on both sides of the first and second cover members 401 and 402 so that the one end 401a of the first cover member 401 and the one end 402a of the second cover member 402 remain in contact with each other.

When the micro needle array 410 is pressed, the elastic supporters 420 provided on both sides are extended and the micro needle array 410 is moved downward. Then, the first and second cover members 401 and 402 provided on the opposite fixed ends 425 of the elastic supporters 420 are moved to the left and right sides respectively so that the one end 401a of the first cover member 401 and the one end 402a of the second cover member 402 are spaced apart from each other. When the elastic supporters 420 is completely extended so that the micro needle array 410 is flushed with the fixed ends 425 of the elastic supporters 420, the first and second cover members 401 and 402 are completely opened so that the micro needle array 410 is exposed through the opening 405 formed between the first and second cover members 401 and 402. Therefore, the exposed micro needle array 410 may be inserted into the patient's skin.

When the needle protection cover 400 is provided below the micro needle array 410 as described above, it is possible to prevent the micro needle array 410 from being exposed to the outside while circulating or handling the bio-signal sensing patch 10.

On the other hand, a micro needle array which can be used for a bio-signal sensing patch according to an embodiment of the present disclosure may be formed to adjust horizontal direction intervals of a plurality of micro needles.

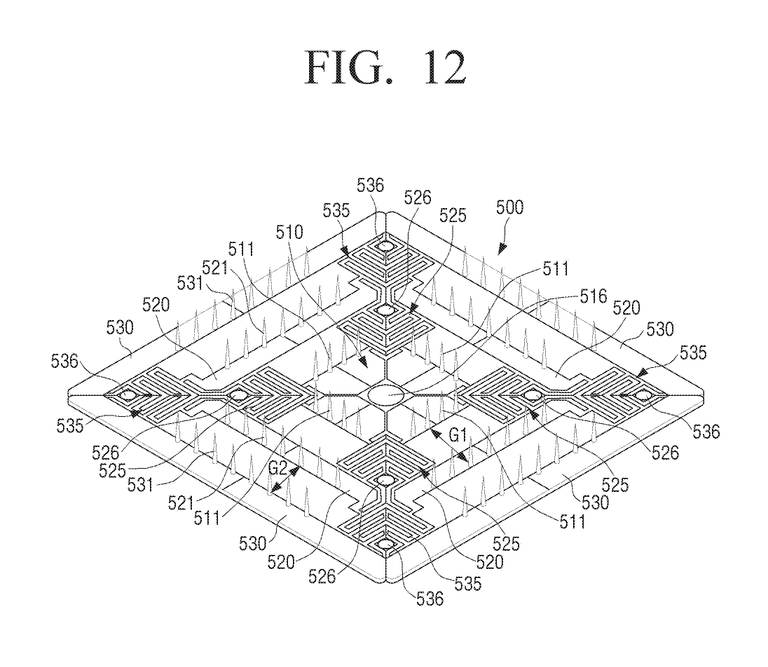

Hereinafter, a structure capable of adjusting intervals between a plurality of micro needles of a micro needle array will be described with reference to FIG. 12.

FIG. 12 is a perspective view illustrating an example of a micro needle array used in a bio-signal sensing patch according to an embodiment of the present disclosure.

Referring to FIG. 12, a micro needle array 500 may include a central array member 510 formed of a rectangular metal plate, four intermediate array members 520 spaced a predetermined distance apart from the four sides of the central array member 510, and four outer array members 530 disposed parallel to and spaced apart a predetermined distance from the four intermediate array members 520. The central array member 510, the four intermediate array members 520, and the four outer array members 530 as described above may be arranged substantially in a square as illustrated in FIG. 12.

On the four sides of the central array member 510, a plurality of micro needles 511 are formed perpendicular to the central array member 510. A plurality of micro needles 521 are provided on the intermediate array member 520 in parallel with the plurality of micro needles 511 provided on one side of the central array member 510. Further, the outer array members 530 are provided with a plurality of micro needles 531 parallel to the plurality of micro needles 521 of the intermediate array member 520, that is, parallel to the plurality of micro needles 511 provided on one side of the central array member 510.

The central array member 510 and the four intermediate array members 520 are connected by four intermediate stretchable portions 525 provided at four corners. Further, the four intermediate array members 520 and the four outer array members 530 are connected by four outer stretchable portions 535 provided at four corners. The intermediate stretchable portions 525 are formed in a spring shape so that the interval between the central array member 510 and the intermediate array members 520 may be adjusted. The outer stretchable portions 535 are also formed in a spring shape so that the intervals between the intermediate array members 520 and the outer array members 530 may be adjusted.

In addition, a center interval adjusting hole 516 is provided at the center of the central array member 510. Intermediate interval adjusting holes 526 are provided at ends of the intermediate stretchable portions 525 adjacent to the outer array members 530. Further, outer interval adjusting holes 536 are provided near the outermost ends of the outer stretchable portions 535. Fixing pins of a needle interval adjusting jig (not illustrated) may be inserted into the center interval adjusting hole 516, the intermediate interval adjusting holes 526, and the outer interval adjusting holes 536.

Therefore, the interval G1 between the plurality of micro needles 511 provided on the central array member 510 and the plurality of micro needles 521 provided on the intermediate array members 520 may be adjusted by inserting the fixing pins of the needle interval adjusting jig into the center interval adjusting hole 516 and the intermediate interval adjusting holes 526 and then moving the intermediate interval adjusting holes 526. For example, when the fixing pins (not illustrated) inserted in the intermediate interval adjusting holes 526 are moved toward the center interval adjusting hole 516, the interval G1 between the micro needles 511 of the central array member 510 and the micro needles 521 of the intermediate array members 520 is narrowed. Conversely, when the fixing pins (not illustrated) inserted in the intermediate interval adjusting holes 526 are moved in the direction away from the center interval adjusting hole 516, the interval G1 between the micro needles 511 of the central array member 510 and the micro needles 521 of the intermediate array members 520 is widened.

In addition, the interval G2 between the plurality of micro needles 521 provided on the intermediate array members 520 and the plurality of micro needles 531 provided on the outer array members 530 may be adjusted by inserting the fixing pins of the needle interval adjusting jig (not illustrated) into the intermediate interval adjusting holes 526 and the outer interval adjusting holes 536 and then moving the outer interval adjusting holes 536. For example, when the fixing pins (not illustrated) inserted in the outer interval adjusting holes 536 are moved toward the intermediate interval adjusting holes 526, the interval G2 between the micro needles 521 of the intermediate array members 520 and the micro needles 531 of the outer array members 530 is narrowed. Conversely, when the fixing pins (not illustrated) inserted in the outer interval adjusting holes 536 are moved in the direction away from the intermediate interval adjusting holes 526, the interval G2 between the micro needles 521 of the intermediate array members 520 and the micro needles 531 of the outer array members 530 is widened.

The micro needle array used in the bio-signal sensing patch according to an embodiment of the present disclosure as described above may be manufactured using a micro electro mechanical system (MEMS) manufacturing process such as an etching process.

The present disclosure has been described above by way example. The terms used herein are for the purpose of description and should not be construed as limiting. Various modifications and variations of the present disclosure are possible in light of the above teachings. Therefore, the present disclosure can be freely carried out within the scope of the claims unless otherwise specified.

What is claimed is:

1. A bio-signal monitoring device comprising:
a sensing patch configured to detect bio-signals and to store the detected bio-signals as data;
a monitoring patch configured to receive the data when approaching the sensing patch; and
an external device configured to receive the data received by the monitoring patch in real time.

2. The bio-signal monitoring device of claim 1, wherein
the bio-signal sensing patch comprises
a sensor to be inserted into a skin and configured to detect the bio-signals;
a memory configured to store the data output from the sensor; and
a short range communicator configured to transmit the data stored in the memory.

3. The bio-signal monitoring device of claim 2, wherein
the bio-signal sensing patch further comprises:
a sensor supporter supporting the sensor and to be in contact with the skin;
a controller disposed on the sensor supporter and configured to control the memory to store the data output from the sensor; and
a power supplier disposed on the sensor supporter and configured to supply power to the sensor, the memory, and the controller.

4. The bio-signal monitoring device of claim 2, wherein
the sensor comprises a micro needle array.

5. The bio-signal monitoring device of claim 2, wherein
the short range communicator comprises a near field communication (NFC) antenna.

6. The bio-signal monitoring device of claim 1, wherein
the monitoring patch comprises a short range communicator configured to receive the data transmitted from the bio-signal sensing patch and to transmit the received data to the external device.

7. The bio-signal monitoring device of claim 6, wherein
the short range communicator comprises a near field communication (NFC) antenna.

8. The bio-signal monitoring device of claim 7, wherein
the short range communicator further comprises one of Bluetooth, wifi, and zigbee.

9. The bio-signal monitoring device of claim 6, wherein
the monitoring patch comprises:
a transmission memory configured to store the data transmitted from the bio-signal sensing patch;
a transmission controller configured to control the short range communicator and the transmission memory to store and transmit the data;
a power supplier configured to supply power to the transmission memory, the short range communicator, and the transmission controller; and
a substrate on which the transmission memory, the short range communicator, the transmission controller, and the power supplier are disposed.

10. The bio-signal monitoring device of claim 9, wherein
the monitoring patch further comprises a display configured to display the data.

11. A bio-signal sensing patch comprising:
a sensor to be inserted into a skin and configured to detect bio-signals;
a sensor supporter supporting the sensor;
a memory disposed on the sensor supporter and configured to store data output from the sensor;
a controller disposed on the sensor supporter and configured to control the memory to store the data output from the sensor;
a short range communicator disposed on the sensor supporter and configured to transmit the data stored in the memory to a reader; and
a power supplier disposed on the sensor supporter and configured to supply power to the sensor, the memory, and the controller,
wherein the short range communicator transmits the data when the reader approaches the short range communicator.

12. The bio-signal sensing patch of claim 11, wherein
the sensor comprises a micro needle array.

13. The bio-signal sensing patch of claim 12, wherein
the micro needle array comprises a plurality of micro needles,
the plurality of micro needles are formed of a shape memory alloy, and
when the micro needles are inserted into the skin, distal end portions of the micro needles are bent obliquely with respect to an inserting direction of the micro needles and prevent the micro needles from falling off from the skin.

14. The bio-signal sensing patch of claim 12, wherein
the micro needle array comprises a plurality of micro needles,
the plurality of micro needles are formed of a bimetal, and
when the micro needles are inserted into the skin, the micro needles are bent obliquely with respect to an inserting direction of the micro needles and prevent the micro needles from falling off from the skin.

15. The bio-signal sensing patch of claim 12, wherein
the sensor supporter comprises at least one elastic bend portion provided on both sides of the micro needle array and defining a protrusion height of the micro needle array.
